(19) United States
(12) Patent Application Publication
(10) Pub. No.: US 2002/0146385 A1
(43) Pub. Date: Oct. 10, 2002

(54) Ionic antimicrobial coating

(76) Inventors: Tung Liang Lin, Acton, MA (US); Min-Shyan Sheu, Chelmsford, MA (US)

Correspondence Address: Y. ROCKY TSAO, Fish & Richardson P.C., 225 Franklin Street, Boston, MA, 02110-2804, US

(21) Appl. No.: 09/829,691

(22) Filed: Apr. 10, 2001

(51) Int. Cl. A61L 2/00; A61K 31/74; B05D 3/00

(52) U.S. Cl. 424/78.27; 427/2.28

(57) ABSTRACT

An antimicrobial coating. The antimicrobial coating contains a water-insoluble polymer having a first ionic group, and an antimicrobial agent having a second ionic group with a charge opposite to that of the first ionic group; wherein the antimicrobial agent is attached to the water-insoluble polymer via an ionic bond between the first ionic group and the second ionic group.

### BACKGROUND OF THE INVENTION

A conventional antimicrobial coating is prepared by physically “entrapping” an antimicrobial agent in a polymer matrix. The antimicrobial agent is released by diffusion at a rate related to several factors, e.g., the solubility and size of the antimicrobial agent, and the pH of the medium in which the antimicrobial coating is placed.

In an aqueous medium, a conventional antimicrobial coating first releases the antimicrobial agent at a high rate and exhibits high antimicrobial activity. The release rate and antimicrobial activity then decrease over time. Thus, a conventional antimicrobial coating is generally effective in preventing microbial growth for only a short period of time.

### SUMMARY OF THE INVENTION

The present invention relates to an antimicrobial coating. The coating includes a water-insoluble polymer and an antimicrobial agent, each of which contains an ionic group. The two ionic groups have opposite charges. The antimicrobial agent is linked to the water-insoluble polymer via an ionic bond between the two ionic groups. The ionic groups mentioned herein refer to those which are substantially ionized or sufficiently polarized in a neutral aqueous solution.

The water-insoluble polymer can be an epoxy polymer, polyester, polyurethane, polyamide, polyacrylamide, poly(acrylic acid), polyphosphazene, or a copolymer thereof. The antimicrobial agent (including antibiotics) can be a biguanide salt, silver salt, polymyxin, tetracycline, aminoglycoside, penicillin, sulfadiazine, rifampicin, bacitracin, neomycin, chloramphenicol, miconazole, nonoxynol 9, fusidic acid, nitrofurazone, norfloxacin, or cephalosporin.

The antimicrobial coating of this invention optionally includes a hydrophilic polymer that is blended with the water-insoluble polymer. Examples of such a hydrophilic polymer include poly(N-vinyl lactam), polyvinylpyrrolidone, polyethyleneoxide, polypropylene oxide, cellulose, polyanhydrate, polyvinyl alcohols, polysaccharide, or polyvinyl ether. The water-insoluble polymer, as well as the water-insoluble polymer and the hydrophilic polymer together, is optionally crosslinked with aziridine, polyfunctional carbodiimide, melamine/urea condensate, or polyfunctional epoxide.

When an antimicrobial coating of this invention is placed in an aqueous medium, the antimicrobial agent, which is ionically bonded to the water-insoluble polymer, is slowly released via ion exchange in a controlled manner. Consequently, effective concentrations of the antimicrobial agent near the coating are maintained for a longer period of time, as compared with a conventional antimicrobial coating.

Details of the invention are set forth in the description below. Other features, objects, and advantages of the invention will be apparent from the description and from the claims.

### DETAILED DESCRIPTION OF THE INVENTION

An antimicrobial coating of this invention can be prepared, for example, by the following method: A water-insoluble polymer that contains ionic groups is first dissolved in a basic aqueous solution. Such a polymer solution can also be prepared by emulsion polymerizing monomers in a basic aqueous solution. If necessary, the pH of the polymer solution is adjusted so that the ionic groups in the polymer are substantially ionized. An antimicrobial agent that also contains ionic groups is then added to the solution. The pH of the solution can be adjusted again, if necessary, for maximal ionization of the polymer and the antimicrobial agent. After gentle stirring for an extended period of time, an antimicrobial coating solution is formed. The coating solution can then be applied to, and form an antimicrobial coating on, a surface of a substrate (e.g., an implantable medical device). For example, a substrate is dipped in the coating solution, removed from it, and then dried. The coating thus obtained renders the substrate surface inhospitable to microorganisms and thereby prevents colonization of bacteria on it. The surface of the substrate, optionally, can be pretreated, e.g., with oxygen plasma, for better adhesion.

The antimicrobial performance of a coating of this invention can be enhanced by including a hydrophilic polymer and a cross-linking agent in the coating solution. For example, the presence of a hydrophilic polymer facilitates the capture of water to create a semi-permanent water zone around the coating, which in turn helps to prevent adhesion of microbes. A cross-linking agent, on the other hand, stabilizes the water-insoluble polymer and further prolongs the release of antimicrobial agents.

The effectiveness of an antimicrobial coating can be determined by conducting a “zone of inhibition” test. In this test, a substrate coated with an antimicrobial coating of this invention is inserted into a lawn of bacteria grown on an agar in such a way that the coating comes in contact with the bacteria. The antimicrobial agent released from the coating effectively inhibits microbial growth in a zone around the coated substrate. The zone, called “zone of inhibition,” is then measured. The size of the zone is an indicator of whether an effective amount of an antimicrobial agent is released from a coating. Conventional coatings release antimicrobial agents in amounts that dramatically decrease over time. In some cases, they become ineffective in only two days. In contrast, antimicrobial coatings disclosed herein, unexpectedly, release antimicrobial agents in effective amounts over up to 60 days.

Without further elaboration, it is believed that one skilled in the art, based on the description herein, can utilize the present invention to its fullest extent. The following specific examples, which describe preparation and uses of several antimicrobial coatings of this invention, are therefore to be construed as merely illustrative, and not limitative of the remainder of the disclosure in any way whatsoever.

### EXAMPLE 1

A 15% aqueous poly(ethylene-co-acrylic acid) (PEA) solution was purchased from Mica Corporation (Stratsford, Conn.). The pH of this solution was 9.2. A 20% aqueous polyvinylpyrrolidone (PVP) solution was prepared by directly dissolving PVP into de-ionized water.

41.67 g of the PEA solution was first diluted with 19.58 g of de-ionized water. To the diluted PEA solution were sequentially added 37.50 g of the PVP solution and 1.00 g of silver chloride. The mixture thus obtained was gently stirred for at least 24 hours until the aqueous phase became saturated with silver chloride, and then filtered through a 50 μm filter to remove excess silver chloride. The filtrate was used as an antimicrobial coating solution.

High-density polyethylene (HDPE) 20 French tubes (0.263×0.229×12″) from Duall Plastics (Athol, Mass.) were treated with oxygen plasma at 100 mTorr and 300 watts for 2 minutes, primed with the 15% PEA solution, and heated at 60° C. for 40 minutes. The tubes were subsequently coated with the antimicrobial coating solution and heated at 60° C. overnight.

The coated tubes were tested in a 30-day release study. In this study, the coated tubes were soaked in artificial urine and collected at five-day intervals. Each of the collected tube was then subjected to an inhibition zone test. See Sawan et al. (Eds) Antimicrobial/Anti-Infective Materials, Chapter 13, 2000, Technomic Publishing Company, Inc., Lancaster, Pa., which is herein incorporated by reference. More specifically, it was vertically inserted into a lawn of Staphylococcus epidermidis grown on an agar for 24 hours in such a way that the coating came in contact with the bacteria. The results show that the sizes of the inhibition zone were unexpectedly the same (2.6 mm) throughout the entire study period.

### EXAMPLE 2

41.67 g of the PEA solution described in Example 1 was diluted with 19.58 g of de-ionized water. To the diluted PEA solution were sequentially added 37.50 g of the PVP solution also described in Example 1 and 1.00 g of silver chloride. After gentle stirring for 24 hours and filtering, 1.25 g of aziridine, a cross-linking agent, was added. The solution thus obtained was further stirred for 30 minutes, resulting in an antimicrobial coating solution.

HDPE 20 French tubes were pretreated with oxygen plasma at 100 mTorr and 300 watts for 2 minutes, primed with the acrylic polymer solution, heated at 60° C. for 40 minutes, coated with the coating solution, and heated again at 60° C. overnight.

The coated tubes were tested in a 30-day release study and following the procedure described in Example 1. The results show that the sizes of the inhibition zones were the same (2.0 mm) throughout the entire study period.

### EXAMPLE 3

A 38% aqueous polyurethane solution (NeoRez R-9621) was purchased from Avecla, Inc. (Wilmington, Mass.).

A priming solution was prepared by mixing 200.00 g of the polyurethane solution, 80.00 g of de-ionized water, and 3.00 g of aziridine.

A coating solution containing aziridine was prepared by the following procedure: 25.00 g of the polyurethane solution was first diluted with 25.00 g of de-ionized water. To the diluted polyurethane solution were sequentially added 13.75 g of the 20% PVP solution described in Example 1 and 0.52 g of silver chloride. The mixture thus obtained was gently stirred for at least 24 hours until the solution became saturated with silver chloride, and filtered through a 50 μm filter to remove excess silver chloride. 0.50 g of aziridine was then added to the filtrate. The solution thus obtained was stirred for another 30 minutes, resulting in an antimicrobial coating solution.

Three more coating solutions were prepared by following the same procedure, except that 0.55 g, 0.575 g, and 0.625 g of aziridine were respectively used.

HDPE 20 French tubes were pretreated with oxygen plasma at 250 mTorr and 250 watts for 2 minutes. The pretreated tubes were subsequently primed with the above-described priming solution, heated at 60° C. for 40 minutes, coated with the four coating solutions, respectively, and heated again at 60° C. overnight.

The coated tubes were tested in a 30-day release study and following the procedure described in Example 1. The results show that the sizes of the inhibition zones of these four coatings were the same (1.85 mm) throughout the entire study period.

### EXAMPLE 4

An antimicrobial coating solution of a different composition was prepared by following the procedure described in Example 3. The solution included 50.0 g of 38% polyurethane solution, 50.0 g of the 20% PVP solution, 60.0 g of de-ionized water, 0.6 g of silver chloride, and 1.0 g of aziridine.

HDPE 20 French tubes were pretreated with oxygen plasma at 100 mTorr and 300 watts for 4 minutes. The tubes were primed with a priming solution including 140.0 g of 38% polyurethane solution, 56.0 g of de-ionized water, and 2.1 g of aziridine, and heated at 65° C. for 30 minutes. The primed tubes were then coated with the antimicrobial coating described above, and heated again at 65° C. for 3 hours.

The coated tubes were tested in a 60-day release study and following the procedure described in Example 1. They were collected at five-day intervals and then used in a zone of inhibition test against staphylococcus epidermidis and Escherichia coli. The results show that the size of inhibition zone remained constant for 50 days (3.0 mm) against Staphylococcus epidermidis and for 60 days (2.0 mm) against Escherichia coli throughout the entire study period.

### OTHER EMBODIMENTS

A number of embodiments of the invention have been described. Nevertheless, it will be understood that various modifications may be made without departing from the spirit and scope of the invention. For example, the antimicrobial coating can be prepared in an organic solvent, instead of water. Accordingly, other embodiments are within the scope of the following claims.

What is claimed is:

1. An antimicrobial coating comprising
a water-insoluble polymer having a first ionic group, and
an antimicrobial agent having a second ionic group with a charge opposite to that of the first ionic group;
wherein the antimicrobial agent is attached to the water-insoluble polymer via an ionic bond between the first ionic group and the second ionic group.

2. The antimicrobial coating of claim 1, wherein the water-insoluble polymer is an epoxy polymer, polyester, polyurethane, polyamide, polyacrylamide, poly(acrylic acid), polyphosphazene, or a copolymer thereof.

3. The antimicrobial coating of claim 2, wherein the antimicrobial agent is biguanide salt, silver salt, polymyxin, tetracycline, aminoglycoside, penicillin, sulfadiazine, rifampicin, bacitracin, neomycin, chloramphenicol, miconazole, nonoxynol 9, fusidic acid, nitrofurazone, norfloxacin, or cephalosporin.

4. The antimicrobial coating of claim 2, wherein the water-insoluble polymer is a polyurethane, poly(acrylic acid), or a copolymer thereof.

5. The antimicrobial coating of claim 4, wherein the antimicrobial agent is biguanide salt, silver salt, sulfadiazine, polymyxin, tetracycline, aminoglycoside, rifampicin, bacitracin, neomycin, chloramphenicol, miconazole, penicillin, nonoxynol 9, fusidic acid, nitrofurazone, norfloxacin, or cephalosporin.

6. The antimicrobial coating of claim 5, further comprising a hydrophilic polymer, the hydrophilic polymer being blended with the water-insoluble polymer.

7. The antimicrobial coating of claim 5, wherein the water-insoluble polymer or the hydrophilic polymer is crosslinked.

8. The antimicrobial coating of claim 6, wherein the water-insoluble polymer or the hydrophilic polymer is crosslinked.

9. The antimicrobial coating of claim 1, wherein the antimicrobial agent is biguanide salt, silver salt, sulfadiazine, polymyxin, tetracycline, aminoglycoside, rifampicin, bacitracin, neomycin, chloramphenicol, miconazole, penicillin, nonoxynol 9, fusidic acid, nitrofurazone, norfloxacin, or cephalosporin.

10. The antimicrobial coating of claim 1, further comprising a hydrophilic polymer, the hydrophilic polymer being blended with the water-insoluble polymer.

11. The antimicrobial coating of claim 10, wherein the water-insoluble polymer or the hydrophilic polymer is crosslinked.

12. The antimicrobial coating of claim 11, wherein the water-insoluble polymer is an epoxy polymer, polyester, polyurethane, polyvinyl chloride, polyamide, polyacrylonitrile, polyacrylamide, poly(acrylic acid), or a copolymer thereof.

13. The antimicrobial coating of claim 11, wherein the antimicrobial agent is biguanide salt, silver salt, polymyxin, tetracycline, aminoglycoside, nitrofurazone, norfloxacin, or penicillin.

14. The antimicrobial coating of claim 12, wherein the antimicrobial agent is biguanide salt, silver salt, sulfadiazine, polymyxin, tetracycline, aminoglycoside, rifampicin, bacitracin, neomycin, chloramphenicol, miconazole, penicillin, nonoxynol 9, fusidic acid, nitrofurazone, norfloxacin, or cephalosporin.

15. The antimicrobial coating of claim 12, wherein the water-insoluble polymer is a polyurethane, poly(acrylic acid), or a copolymer thereof.

16. The antimicrobial coating of claim 15, wherein the antimicrobial agent is biguanide salt, silver salt, sulfadiazine, polymyxin, tetracycline, aminoglycoside, rifampicin, bacitracin, neomycin, chloramphenicol, miconazole, penicillin, nonoxynol 9, fusidic acid, nitrofurazone, norfloxacin, or cephalosporin.

17. The antimicrobial coating of claim 16, wherein the antimicrobial agent is biguanide salt, silver salt, polymyxin, aminoglycoside, nitrofurazone, or norfloxacin.

18. The antimicrobial coating of claim 17, wherein the hydrophilic polymer is a poly(N-vinyl lactam), polyvinylpyrrolidone, polyethyleneoxide, polypropylene oxide, cellulose, polyanhydrate, polyvinyl alcohols, polysaccharide, or polyvinyl ether.

19. The antimicrobial coating of claim 17, wherein the crosslinking agent is aziridine, polyfunctional carbodiimide, melamine/urea condensate, or polyfunctional epoxide.

20. The antimicrobial coating of claim 18, wherein the crosslinking agent is aziridine, polyfunctional carbodiimide, melamine/urea condensate, or polyfunctional epoxide.

21. The antimicrobial coating of claim 20, wherein the hydrophilic polymer is polyvinylpyrrolidone.

22. The antimicrobial coating of claim 21, wherein the crosslinking agent is aziridine.

23. The antimicrobial coating of claim 22, wherein the water-insoluble polymer is an acrylic copolymer.

24. The antimicrobial coating of claim 23, wherein the antimicrobial agent is silver chloride.

25. The antimicrobial coating of claim 23, wherein the antimicrobial agent is silver oxide.

26. The antimicrobial coating of claim 22, wherein the water-insoluble polymer is a polyurethane.

27. The antimicrobial coating of claim 26, wherein the antimicrobial agent is silver chloride.

28. The antimicrobial coating of claim 26, wherein antimicrobial agent (A) is silver chloride and antimicrobial agent (B) is norfloxacin.

29. The antimicrobial coating of claim 26, wherein antimicrobial agent (A) is silver chloride, antimicrobial agent (B) is norfloxacin, and antimicrobial agent (C) is miconazole nitrate.

30. The antimicrobial coating of claim 1, wherein the water-insoluble polymer is crosslinked.
